SEQUENCE LISTING (38 sequences)

SEQ ID NO: 1 - DNA, 21 bp, Artificial Sequence
  Human SNP in Sp4 Gene (rs12673091)
tagggtactcatgaaaagact

SEQ ID NO: 2 - DNA, 21 bp, Artificial Sequence
  Human SNP in Sp4 Gene (rs12673091)
tagggtactcgtgaaaagact

SEQ ID NO: 3 - DNA, 21 bp, Artificial Sequence
  Human SNP in Sp4 Gene (rs10245440)
tacttatctaataaacacagc

SEQ ID NO: 4 - DNA, 21 bp, Artificial Sequence
  Human SNP in Sp4 Gene (rs10245440)
tacttatctactaaacacagc

SEQ ID NO: 5 - DNA, 21 bp, Artificial Sequence
  Human SNP in Sp4 gene (rs10261327)
tctcagttaacacacttttga

SEQ ID NO: 6 - DNA, 21 bp, Artificial Sequence
  Human SNP in Sp4 gene (rs10261327)
tctcagttaatacacttttga

SEQ ID NO: 7 - DNA, 21 bp, Artificial Sequence
  Human SNP in Sp4 gene (rs40245)
tcctaattctaaggtttgacg

SEQ ID NO: 8 - DNA, 21 bp, Artificial Sequence
  Human SNP in Sp4 gene (rs40245)
tcctaattcttaggtttgacg

SEQ ID NO: 9 - DNA, 21 bp, Artificial Sequence
  Human SNP in Sp4 gene (rs2282888)
tgtttttactatttgaagtga

SEQ ID NO: 10 - DNA, 21 bp, Artificial Sequence
  Human SNP in Sp4 gene (rs2282888)
tgtttttactgtttgaagtga

SEQ ID NO: 11 - DNA, 21 bp, Artificial Sequence
  Human SNP in Sp4 gene (rs10276352)
ttttagagaaatttcaagtgt

SEQ ID NO: 12 - DNA, 21 bp, Artificial Sequence
  Human SNP in Sp4 gene (rs10276352)
ttttagagaagtttcaagtgt

SEQ ID NO: 13 - DNA, 21 bp, Artificial Sequence
  Human SNP in Sp4 gene (rs12668354)
ggagatactcacctaaatgtc

SEQ ID NO: 14 - DNA, 21 bp, Artificial Sequence
  Human SNP in Sp4 gene (rs12668354)
ggagatactcccctaaatgtc

SEQ ID NO: 15 - DNA, 21 bp, Artificial Sequence
  Human SNP in Sp4 gene (rs1018954)
ctaacagttaatcattgtgca

SEQ ID NO: 16 - DNA, 21 bp, Artificial Sequence
  Human SNP in Sp4 gene (rs1018954)
ctaacagttattcattgtgca

SEQ ID NO: 17 - DNA, 21 bp, Artificial Sequence
  Human SNP in Sp4 gene (rs11974306)
ttttgtgggtatatgtgtgga

SEQ ID NO: 18 - DNA, 21 bp, Artificial Sequence
  Human SNP in Sp4 gene (rs11974306)
ttttgtgggtttatgtgtgga

SEQ ID NO: 19 - DNA, 21 bp, Artificial Sequence
  Human SNP in CACNG2 (rs4820239)
gggaccttccactactccctc

SEQ ID NO: 20 - DNA, 21 bp, Artificial Sequence
  Human SNP in CACNG2 (rs4820239)
gggaccttccgctactccctc

SEQ ID NO: 21 - DNA, 21 bp, Artificial Sequence
  Human SNP in CACNG2 (rs2267341)
cattcagacacagacactcaa

SEQ ID NO: 22 - DNA, 21 bp, Artificial Sequence
  Human SNP in CACNG2 (rs2267341)
cattcagacatagacactcaa

SEQ ID NO: 23 - DNA, 21 bp, Artificial Sequence
  Human SNP in CACNG2 (rs2283981)
tgacatgccccgaggcgtccc

SEQ ID NO: 24 - DNA, 21 bp, Artificial Sequence
  Human SNP in CACNG2 (rs2283981)
tgacatgccctgaggcgtccc

SEQ ID NO: 25 - DNA, 21 bp, Artificial Sequence
  Human SNP in CACNG2 (rs3788521)
tctctcgttccttcattcatt

SEQ ID NO: 26 - DNA, 21 bp, Artificial Sequence
  Human SNP in CACNG2 (rs3788521)
tctctcgttcgttcattcatt

SEQ ID NO: 27 - DNA, 21 bp, Artificial Sequence
  Human SNP in CACNG2 (rs738977)
atgaaatacccgcagccggta

SEQ ID NO: 28 - DNA, 21 bp, Artificial Sequence
  Human SNP in CACNG2 (rs738977)
atgaaatacctgcagccggta

SEQ ID NO: 29 - DNA, 21 bp, Artificial Sequence
  Human SNP in CACNG2 (rs738518)
agacaaagggcgagtgacttc

SEQ ID NO: 30 - DNA, 21 bp, Artificial Sequence
  Human SNP in CACNG2 (rs738518)
agacaaagggtgagtgacttc

SEQ ID NO: 31 - DNA, 21 bp, Artificial Sequence
  Human SNP in CACNG2 (rs200966)
tggatcacgaagtcaggagtt

SEQ ID NO: 32 - DNA, 21 bp, Artificial Sequence
  Human SNP in CACNG2 (rs200966)
tggatcacgaggtcaggagtt

SEQ ID NO: 33 - DNA, 21 bp, Artificial Sequence
  Human SNP in CACNG2 (rs3484)
ggagggtggcaagaggggccg

SEQ ID NO: 34 - DNA, 21 bp, Artificial Sequence
  Human SNP in CACNG2 (rs3484)
ggagggtggcgagaggggccg

SEQ ID NO: 35 - DNA, 21 bp, Artificial Sequence
  Human SNP in CACNG2 (rs736720)
catcctcgtcagctctcttat

SEQ ID NO: 36 - DNA, 21 bp, Artificial Sequence
  Human SNP in CACNG2 (rs736720)
catcctcgtcggctctcttat

SEQ ID NO: 37 - DNA, 21 bp, Artificial Sequence
  Human SNP in CACNG2 (rs2284026)
cagctcactccaggagcctag

SEQ ID NO: 38 - DNA, 21 bp, Artificial Sequence
  Human SNP in CACNG2 (rs2284026)
cagctcactctaggagcctag

(19) United States
(12) Patent Application Publication
(10) Pub. No.: US 2010/0222415 A1
(43) Pub. Date: Sep. 2, 2010

(54) Method to Diagnose, Predict Treatment Response and Develop Treatment for Psychiatric Disorders Using Markers

(75) Inventors: John R. Kelsoe, Del Mar, CA (US); Sherri Liang, San Diego, CA (US); Xianjin Zhou, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: The Regents of the University of California, Oakland, CA (US)

(21) Appl. No.: 11/992,835

(22) PCT Filed: Oct. 13, 2006
(86) PCT No.: PCT/US06/40475

§ 371 Date: Mar. 28, 2008

Related U.S. Application Data
(60) Provisional application No. 60/726,978, filed on Oct. 14, 2005.
(60) Provisional application No. 60/749,668, filed on Dec. 9, 2005.

(51) Int. Cl. A61K 31/7088; C12Q 1/68; G01N 33/567; C07H 21/04; A61P 25/18

(52) U.S. Cl. 514/44 R; 435/6; 435/7.21; 536/23.1

(57) ABSTRACT

The disclosure provides methods and compositions useful for identifying a subject's predisposition or diagnosis of a mental disorder, methods of screening for agents useful for treating such a disorder and methods of treatment thereof.

### CROSS REFERENCE TO RELATED APPLICATIONS

The application claims priority under 35 U.S.C. §119 to U.S. Provisional Application Ser. Nos. 60/726,978, filed Oct. 14, 2005, and 60/749,668, filed Dec. 9, 2005, the disclosures of which are incorporated herein by reference.

### STATEMENT REGARDING FEDERALLY SPONSORED RESEARCH

This invention was supported in part by Grant Nos. MH47612, MH59567 and MH68503 awarded by the National Institutes of Health. The government may have certain rights in this invention.

### TECHNICAL FIELD

The invention relates to predicting the probability that a subject will present with or has a mental disorder.

### BACKGROUND

Presently, there are no biological tests to aid in the diagnosis of psychiatric disorders. Diagnosis is, therefore, largely based on behavior rather than underlying biology or pathophysiology. Accurate diagnosis frequently requires years for the longitudinal course of symptoms to be clear. Current behavior-based diagnoses have limited ability to predict course or response to treatment. A genetic test for pathogenic mutations in the genome will enable more rapid and more accurate diagnosis, thereby leading to better treatment. No such test currently exists. There is currently only a limited understanding of the biological mechanisms of most psychiatric disorders.

### SUMMARY

The invention provides a method and compositions useful to diagnose, prognose, treat, and screen agents associated with mental disorders.

The invention provides a method comprising providing a sample comprising polynucleotides obtained from a subject; contacting the sample with at least one probe comprising an oligonucleotide that interacts with a single nucleotide polymorphism (SNP) in CACNG2 and/or in Sp4 polynucleotide; and detecting the binding of the at least one probe to a polynucleotide in the sample. The SNP is selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 and any combination thereof.

The invention also provides a method for determining whether a subject comprises a mental or mood disorder, comprising contacting a sample with at least one probe comprising at least 8 contiguous nucleotides of SEQ ID NO:1-37 or 38, and containing nucleotide 11 or the complement thereof; and determining if the sample comprises a polynucleotide molecule that hybridizes to the probe. In one aspect, the invention further comprises diagnosing a mental disorder or clinical symptom in the subject selected from the group consisting of euphoric mania, dysphoric mania, Bipolar I, Rapid Cycling, History of Suicide Attempt, PTSD, Panic Attacks/Panic Disorder, Alcohol or Substance Dependence, and any combination thereof.

The invention includes a method of determining a subject's predisposition to a mental disorder comprising detecting at least one polymorphism in a gene selected from the group consisting of CACNG2 and Sp4, wherein the polymorphism is selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 and any combination thereof.

The invention further includes a method of diagnosing a subject for a mental disorder comprising detecting at least one polymorphism in a gene selected from the group consisting of CACNG2 and Sp4, wherein the polymorphism is selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs200966, rs3484, rs736720, rs2284026 and any combination thereof.

The methods used in detecting a polymorphism can include, for example, a primer extension assay; an allele-specific PCR assay; a nucleic acid amplification assay; a hybridization assay; a mismatch-detection assay; an enzymatic nucleic acid cleavage assay; and a sequencing assay.

The invention also include methods of screening for agents useful for treating a mental disorder associated with a polymorphism in a CACNG2 and/or Sp4 gene comprising contacting a cell that expresses a polymorphism selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 with a test agent; and detecting interaction of the agent with the CACNG2 or Sp4 gene product. In one aspect, the agent can inhibit or increase expression of a CACNG2 or Sp4 gene product.

The invention also includes oligonucleotides, kits, devices, and substrates useful detecting a polymorphism associated with a mental disorder. In one aspect, an oligonucleotide is comprises a sequence selected from the group consisting of SEQ ID NO:1-37 or 38 and containing nucleotide 11. The oligonucleotide can be removably associated with or bonded to a solid support.

The invention includes a method of treating a subject with a mental disorder, comprising contacting a subject containing a mutation in a CACNG2 or Sp4 gene with an agent that inhibits production of expression or activity of the mutant gene.

The invention yet further includes a method of treating a subject with a mental disorder, comprising contacting a subject containing a mutation in a CACNG2 or Sp4 gene with a polynucleotide comprising a wild type CACNG2 or Sp4 under conditions wherein the wild type CACNG2 or Sp4 is expressed in vivo.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1 is a Phase 1 High density SNP mapping study of Bipolar Disorder and 22q13 implicates the CACNG2 gene.

FIG. 2 is a plot depicting association of Bipolar Disorder to the CACNG2 Gene in an Independent Sample.

FIG. 3 depicts a summary of bipolar and schizophrenia loci on chromosome 22.

FIG. 4 shows a schematic of chromosome 22 and a genome survey of 164 subjects from 20 families.

FIG. 5 shows a proposed mechanism of CACNG2 activity.

FIG. 6 shows findings near locus D22S278 on Chromosome 22.

FIG. 7 is a histological analysis of hippocampi in the Sp4 null mutant mice. Sagittal sections of both wild-type and Sp4 KO mouse hippocampi, at postnatal Day 3 and Day 7 respectively, stained by Hematoxylin-Eosin. G: dentate granule layer. Scale Bars are 200 μm.

FIG. 8A-F show characterization of the developing hippocampal dentate gyrus at postnatal Day 3. Sagittal sections of both wild type and Sp4 KO mouse hippocampi, at postnatal Day3, were analyzed by immunohistochemical staining with anti-Prox-1 (a, b), and anti-Phospho-Histone-H3 (c, d). The mitotic index revealed by immunohistochemical staining with anti-Phospho-Histone-H3 in cerebellum (e, f). Wild type siblings (a, c, e). Sp4 KO mice (b, d, f). Scale Bars are 200 μm. Two tailed t-test was used for statistical analysis. *p<0.05.

FIG. 9 shows dendritic growth of neonatal mouse dentate granule neurons in in vitro culture. Identification of dentate granule neurons from hippocampal culture by immunostaining with anti-Prox-1. The dendrites of granule neurons were revealed by coimmunostaining with anti-MAP2. About 50 to 60 dentate granule cells were randomly selected from both wild type and Sp4 KO cultures, respectively. The dendritic length was measured by using Trace Program in Image-Pro Plus software. Two tailed t-test was used for statistical analysis. ***p<0.01.

FIG. 10A-F show histological and stereological analysis of adult mouse hippocampus. Sagittal sections of both wild type and Sp4 KO adult mouse hippocampi stained by Hematoxylin-Eosin (a-f). Wild type siblings (a, c, e). Sp4 KO mice (b, d, f). Stereological analysis of four pairs of mouse hippocampi was summarized in the graph. G: dentate granule layer. Sale Bars are 100 μm (a, b, c, d), 30 μm (e, f). Two tailed t-test was used for statistical analysis. *p<0.05.

FIG. 11A-D shows characterization of adult mouse hippocampus. Expression of Scip gene in hippocampal CA1 region by RNA in situ hybridization (a, b). Immunohistochemical analysis of synaptophysin expression in the molecular layer of dentate granule layer (c, d). Wildtype mice (a, c). Sp4 KO mice (b, d). G: dentate granule layer. Scale Bars are 100 μm (a, b), 50 μm (c, d).

FIG. 12 depicts the arrangement of SNPs in Sp4.

### DETAILED DESCRIPTION

As used herein and in the appended claims, the singular forms “a,” “and,” and “the” include plural referents unless the context clearly dictates otherwise. Thus, for example, reference to “a polynucleotide” includes a plurality of such polynucleotides and reference to “the SNP” includes reference to one or more SNPs known to those skilled in the art, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

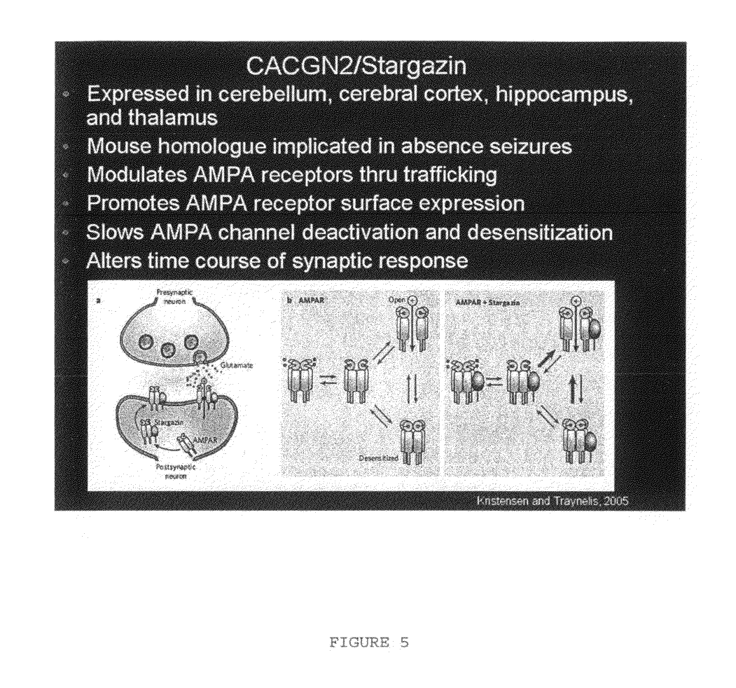

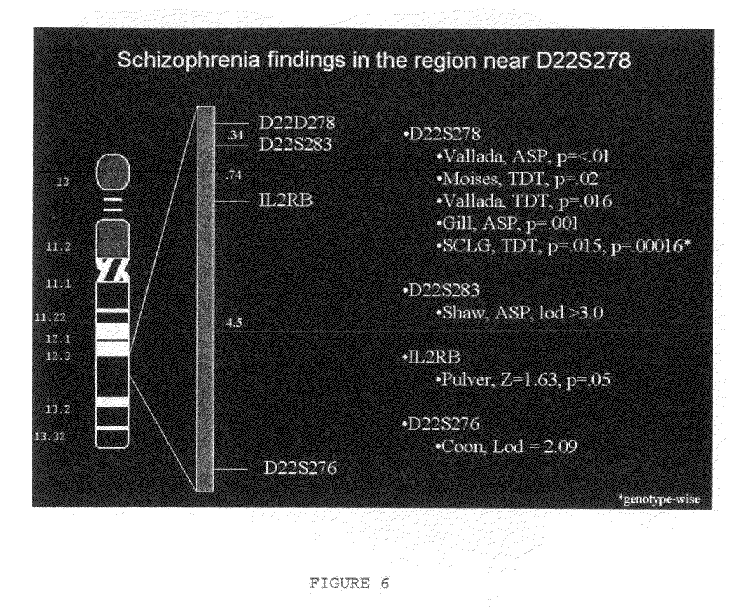

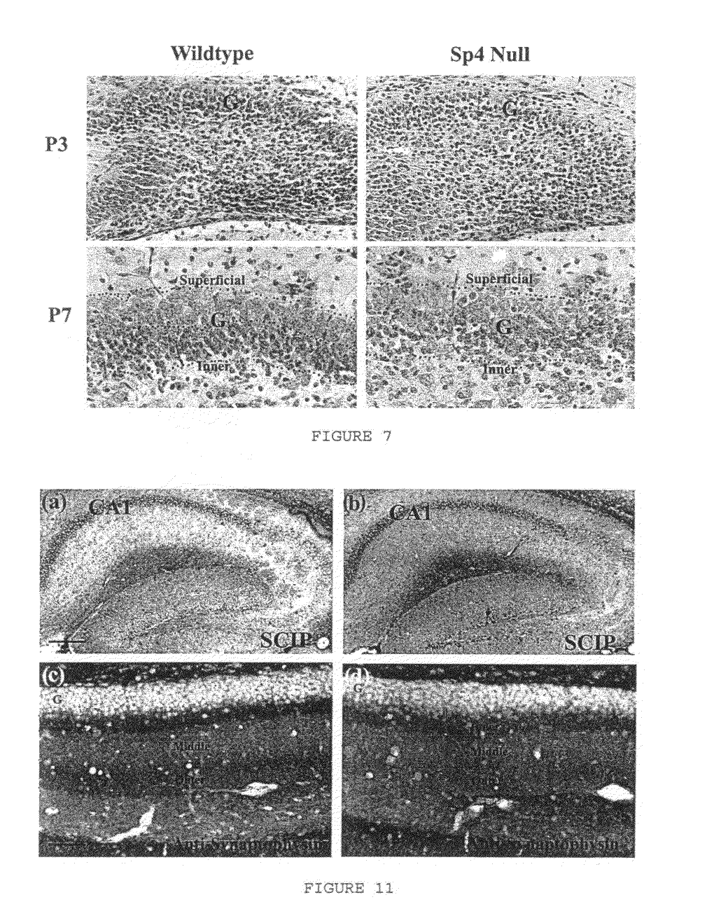

AMPA-type glutamate receptors mediate the majority of the fast excitatory synaptic transmission and contribute to synaptic flexibility in the brain. Stargazin (also teamed CACNG2) is a member of a recently identified protein family termed transmembrane AMPA receptor regulatory proteins (TARPS). These proteins physically associate with AMPA receptors and participate in their surface delivery and anchoring at the postsynaptic membrane (see, e.g., FIG. 5). Thus, these proteins are associated with neuronal activity.

The invention demonstrates a link between mutations in the Sp4 and CACNG2 genes and mental or psychiatric disorders. A DNA test for pathogenic mutations in a Sp4 gene and/or a CACNG2 gene will enable more rapid and more accurate diagnosis, thereby leading to better treatment. The SP4 and/or CACNG2 gene, polynucleotides and fragments thereof can be used to develop polynucleotide-based tests that can be used as diagnostics and prognostics and can also be used to predict responses to medications. No such test currently exists. Furthermore, there is currently only a limited understanding of the biological mechanisms of most psychiatric disorders. This results in an inability to select novel targets for the development of therapeutics that have a different mechanism of action. No current therapeutics target the Sp4 or CACNG2 gene or gene product or pathway.

The invention provides methods, compositions, and devices for determining whether a subject has or is predisposed for a mental or psychiatric disorder (e.g., a bipolar disorder). In one embodiment, the method comprises (i) obtaining a biological sample from a subject; (ii) contacting the sample with a reagent that selectively associates with a polynucleotide or polypeptide indicative of a polymorphism or mutation in an Sp4 and/or CACNG2 (Stargazin) gene; and (iii) detecting the level of reagent that selectively associates with the sample, thereby determining whether the subject has or is predisposed for a mental or psychiatric disorder. In one aspect, the reagent comprises an oligonucleotide that selectively hybridizes to a polymorphic region of an Sp4 gene or CACNG2 (Stargazin) gene under stringent conditions. In another embodiment, the reagent comprises an antibody that selectively binds to a mutant Sp4 or Stargazin gene product.

By employing an SP4 and/or CACNG2 polynucleotide of the invention as a predictor of psychiatric disorders, the invention can, for example, 1) aid in the diagnosis of neuropsychiatric illnesses by the use of a polynucleotide based test; 2) aid in the selection of medication for subjects with neuropsychiatric illness by the use of a polynucleotide based test; 3) develop a screening test for compounds that bind to or affect the function of a SP4 and/or CACNG2 polynucleotide that may be of therapeutic utility in neuropsychiatric illness; and 4) be used as a target for gene therapy or other gene of DNA based therapeutic approaches to neuropsychiatric illnesses.

The invention provides SNPs and haplotypes in the Sp4 and CACNG2 gene that are associated with psychiatric disorders (e.g., bipolar disorder). These SNPs can be used to aid in diagnosis. As additional mutations are found, the power and usefulness of the test will improve. In addition, the application of the SNPs from other genes in combination with the invention can assist in the diagnosis of other neuropsychiatric disorders, such as depression or schizophrenia. Similarly, drug response in these disorders may be tested for association to SNPs in the Sp4 and CACNG2 genes to assist in predicting drug response.

The invention demonstrates a link between mutations in the CACNG2 gene and psychiatric disorders. In one embodiment, an oligonucleotide test for mutations in a CACNG2 gene, will thus enable more rapid and more accurate diagnosis, thereby leading to better treatment. The CACNG2 gene, polynucleotides and fragments thereof can be used to develop oligonucleotide-based tests.

The invention demonstrates a link between mutations in the Sp4 gene and psychiatric disorders. In one embodiment, an oligonucleotide test for mutations in an Sp4 gene, will thus enable more rapid and more accurate diagnosis, thereby leading to better treatment. The Sp4 gene, polynucleotides and fragments thereof can be used to develop oligonucleotide-based tests.

In one embodiment of the invention, diagnosis of a susceptibility to psychiatric disorders is made by detecting a polymorphism in an Sp4 and/or CACNG2. The polymorphism can be a mutation in Sp4 and/or a CACNG2, such as the insertion or deletion of a single nucleotide, or of more than one nucleotide. In some instances the change of, insertion of or deletion of a nucleotide can result in a change in the encoded Sp4 or CACNG2 gene product. Thus, a polymorphism can be detected in the gene sequence or in the encoded gene product. More than one such mutation may be present in a single gene. For example, if the mutation is a frame shift mutation, the frame shift can result in a change in the encoded amino acids, and/or can result in the generation of a premature stop codon, causing generation of a truncated polypeptide. Alternatively, a polymorphism associated with a susceptibility to psychiatric disorders can be a synonymous mutation in one or more nucleotides (i.e., a mutation that does not result in a change in the polypeptide). Such a polymorphism may alter splicing sites, affect the stability or transport of mRNA, or otherwise affect the transcription or translation of the gene. An Sp4 or CACNG2 that has any of the mutations described above is referred to herein as a “mutant gene.”

In one method of the invention, polynucleotides (e.g., DNA) are obtained from a subject. The subject's polynucleotides are then used in a test for one or more SNPs including:

TABLE 1

| Gene | rs# | Sequence | SEQ ID NO: |
|---|---|---|---|
| Sp4 | rs12673091 | TAGGGTACTC[A/G]TGAAAAGACT | 1 and 2 |
| Sp4 | rs10245440 | TACTTATCTA[A/C]TAAACACAGC | 3 and 4 |
| Sp4 | rs10261327 | TCTCAGTTAA[C/T]ACACTTTTGA | 5 and 6 |
| Sp4 | rs40245 | TCCTAATTCT[A/T]AGGTTTGACG | 7 and 8 |
| Sp4 | rs2282888 | TGTTTTTACT[A/G]TTTGAAGTGA | 9 and 10 |
| Sp4 | rs10276352 | TTTTAGAGAA[A/G]TTTCAAGTGT | 11 and 12 |
| Sp4 | rs12668354 | GGAGATACTC[A/C]CCTAAATGTC | 13 and 14 |
| Sp4 | rs1018954 | CTAACAGTTA[A/T]TCATTGTGCA | 15 and 16 |
| Sp4 | rs11974306 | TTTTGTGGGT[A/T]TATGTGTGGA | 17 and 18 |
| CACNG2 (region1) | rs4820239 | GGGACCTTCC[A/G]CTACTCCCTC | 19 and 20 |
| CACNG2 (region1) | rs2267341 | CATTCAGACA[C/T]AGACACTCAA | 21 and 22 |
| CACNG2 (region1) | rs2283981 | TGACATGCCC[C/T]GAGGCGTCCC | 23 and 24 |
| CACNG2 (region1) | rs3788521 | TCTCTCGTTC[C/G]TTCATTCATT | 25 and 26 |
| CACNG2 (region1) | rs738977 | ATGAAATACC[C/T]GCAGCCGGTA | 27 and 28 |
| CACNG2 (region2) | rs738518 | AGACAAAGGG[C/T]GAGTGACTTC | 29 and 30 |
| CACNG2 (region2) | rs2009667 | cctttgggag[A/G]agagaaaggaa | 31 and 32 |
| CACNG2 (region2) | rs3484 | GGAGGGTGGC[A/G]AGAGGGGCCG | 33 and 34 |
| CACNG2 (region2) | rs736720 | CATCCTCGTC[A/G]GCTCTCTTAT | 35 and 36 |
| CACNG2 (region2) | rs2284026 | CAGCTCACTC[C/T]AGGAGCCTAG | 37 and 38 |

If a subject has one or more of the SNPs identified above (e.g., at least rs12673091) then the subject has, or is predisposed to having, a psychiatric disorder such as, but not limited to, a bipolar disorder.

In the context of this disclosure, the terms below shall be defined as follows unless otherwise indicated:

An allele is a particular form of a genetic locus, distinguished from other forms by its particular nucleotide sequence, or one of the alternative polymorphisms found at a polymorphic site.

A gene refers to a segment of genomic DNA that contains the coding sequence for a protein, wherein the segment may include promoters, exons, introns, and other untranslated regions that control expression.

A genotype is an unphased 5′ to 3′ sequence of nucleotide pair(s) found at a set of one or more polymorphic sites in a locus on a pair of homologous chromosomes in an individual. As used herein, genotype includes a full-genotype and/or a sub-genotype.

Genotyping is a process for determining a genotype of an individual.

A haplotype is a 5′ to 3′ sequence of nucleotides found at a set of one or more polymorphic sites in a locus on a single chromosome from a single individual.

Haplotype pair is two haplotypes found for a locus in a single individual.

Haplotyping is the process for determining one or more haplotypes in an individual and includes use of family pedigrees, molecular techniques and/or statistical inference.

A genetic locus refers to a location on a chromosome or DNA molecule corresponding to a gene or a physical or phenotypic feature, where physical features include polymorphic sites.

A “mental disorder” or “mental illness” or “mental disease” or “psychiatric or neuropsychiatric disease or illness or disorder” refers to mood disorders (e.g., major depression, mania, and bipolar disorders), psychotic disorders (e.g., schizophrenia, schizoaffective disorder, schizophreniform disorder, delusional disorder, brief psychotic disorder, and shared psychotic disorder), personality disorders, anxiety disorders (e.g., obsessive compulsive disorder) as well as other mental disorders such as substance-related disorders, childhood disorders, dementia, autistic disorder, adjustment disorder, delirium, multi-infarct dementia, and Tourette's disorder.

“A psychotic disorder” refers to a condition that affects the mind, resulting in at least some loss of contact with reality. Symptoms of a psychotic disorder include, e.g., hallucinations, changed behavior that is not based on reality, delusions and the like. See, e.g., DSM IV. Schizophrenia, schizoaffective disorder, schizophreniform disorder, delusional disorder, brief psychotic disorder, substance induced psychotic disorder, and shared psychotic disorder are examples of psychotic disorders.

“Schizophrenia” refers to a psychotic disorder involving a withdrawal from reality by an individual. Symptoms include: delusions; hallucinations; disorganized speech; grossly disorganized or catatonic behavior; or negative symptoms, i.e., affective flattening, alogia, or avolition.

A “mood disorder” refers to disruption of feeling tone or emotional state experienced by an individual for an extensive period of time. Mood disorders include major depression disorder (i.e., unipolar disorder), mania, dysphoria, bipolar disorder, dysthymia, and cyclothymia.

“Bipolar disorder” is a mood disorder characterized by alternating periods of extreme moods. Bipolar disorders include bipolar disorder I (mania with or without major depression) and bipolar disorder II (hypomania with major depression).

Polymorphic site (PS) a position on a chromosome or DNA molecule at which at least two alternative sequences are found in a population.

A polymorphism refers to the sequence variation observed in an individual at a polymorphic site. Polymorphisms include nucleotide substitutions, insertions, deletions and microsatellites and may, but need not, result in detectable differences in gene expression or protein function. A single nucleotide polymorphism (SNP) is a single change in the nucleotide variation at a polymorphic site.

An oligonucleotide probe or a primer refers to a nucleic acid molecule of between 8 and 2000 nucleotides in length, or is specified to be about 6 and 1000 nucleotides in length. More particularly, the length of these oligonucleotides can range from about 8, 10, 15, 20, or 30 to 100 nucleotides, but will typically be about 10 to 50 (e.g., 15 to 30 nucleotides). The appropriate length for oligonucleotides in assays of the invention under a particular set of conditions may be empirically determined by one of skill in the art.

Oligonucleotide primers and probes can be prepared by any suitable method, including, for example, cloning and restriction of appropriate sequences and direct chemical synthesis.

Oligonucleotide probes and primers can comprise nucleic acid analogs such as, for example peptide nucleic acids, locked nucleic acid (LNA) analogs, and morpholino analogs. The 3′ end of the probe can be functionalized with a capture or detectable label to assist in detection of a polymorphism.

Any of the oligonucleotides or nucleic acid of the invention can be labeled by incorporating a detectable label measurable by spectroscopic, photochemical, biochemical, immunochemical, or chemical means. For example, such labels can comprise radioactive substances (³²P, ³⁵S, ³H, ¹²⁵I) fluorescent dyes (5-bromodesoxyuridin, fluorescein, acetylaminofluorene, digoxigenin), biotin, nanoparticles, and the like. Such oligonucleotides are typically labeled at their 3′ and 5′ ends.

A reference or control population refers to a group of subjects or individuals who are predicted to be representative of the genetic variation found in the general population. Typically, the reference population represents the genetic variation in the population at a certainty level of at least 85%, typically at least 90%, least 95% and but commonly at least 99%. Typically the control population refers to a group that lacks any clinical sign of a mental or psychiatric disorder.

A subject comprises an individual (e.g., a mammalian subject or human) whose genotypes or haplotypes, disease or disorder, or response to treatment or disease state are to be determined.

A probe refers to a molecule which can detectably distinguish between target molecules differing in structure. Detection can be accomplished in a variety of different ways depending on the type of probe used and the type of target molecule. Thus, for example, detection may be based on discrimination of activity levels of the target molecule, but typically is based on detection of specific binding. Examples of such specific binding include antibody binding and oligonucleotide probe hybridization, amplification techniques or others described below. Thus, for example, probes can include enzyme substrates, antibodies and antibody fragments, oligonucleotide hybridization probes and oligonucleotide primers. Thus, in one embodiments, the detection of the presence or absence of the at least one mutant, variant, polymorphism involves contacting a target polymorphic site with a probe, typically an oligonucleotide probe, where the probe binds or hybridizes with a form of the target polymorphic site (e.g., the target nucleic acid containing a complementary base at the variance site as compared to hybridization to a form of the target nucleic acid having a non-complementary base at the variance site, where the hybridization is carried out under selective hybridization conditions). An oligonucleotide probe may span two or more variance sites. Unless otherwise specified, an oligonucleotide probe can include one or more nucleic acid analogs, labels or other substituents or moieties so long as the base-pairing function is retained.

The SNPs identified herein can be used in combination with additional predictive tests including, but not limited to, additional SNPs, mutations, and clinical tests. For example, oligonucleotide probes of the invention comprise at least 8 nucleotides of SEQ ID NOs:1-37, and/or 38 containing the underlined sequence above (including any of the foregoing sequences wherein T can be U and any complements of any of the foregoing sequences) and wherein the oligonucleotide hybridizes to a polynucleotide sample from a subject comprising SEQ ID NO:1-38. In one aspect, an oligonucleotide probe of the invention comprises SEQ ID NO:1-37 or 38 having at least 8 nucleotide and containing nucleotides 10-12 (e.g., from about nucleotide 6 to nucleotide 14 of SEQ ID NO:1-37 or 38).

As mentioned above, such probes can comprise nucleotide analogs useful for hybridization such as Locked Nucleic Acids (LNA). As described herein, such probes can be immobilized on a substrate such as a gene chip.

Any of the oligonucleotide primers and probes of the invention can be immobilized on a solid support. Solid supports are known to those skilled in the art and include the walls of wells of a reaction tray, test tubes, polystyrene beads, magnetic beads, nitrocellulose strips, membranes, microparticles such as latex particles, glass and the like. The solid support is not critical and can be selected by one skilled in the art. Thus, latex particles, microparticles, magnetic or non-magnetic beads, membranes, plastic tubes, walls of microtiter wells, glass or silicon chips and the like are all suitable examples. Suitable methods for immobilizing oligonucleotides on a solid phase include ionic, hydrophobic, covalent interactions and the like. The solid support can be chosen for its intrinsic ability to attract and immobilize the capture reagent. The oligonucleotide probes or primers of the invention can be attached to or immobilized on a solid support individually or in groups of about 2-10,000 distinct oligonucleotides of the invention to a single solid support.

A substrate comprising a plurality of oligonucleotide primers or probes of the invention may be used either for detecting or amplifying targeted sequences in polymorphic region of Sp4 and/or CACNG2 genes based upon the polymorphic sequences above.

The oligonucleotide probes and primers of the invention can be attached in contiguous regions or at random locations on the solid support. Alternatively the oligonucleotides of the invention may be attached in an ordered array wherein each oligonucleotide is attached to a distinct region of the solid support which does not overlap with the attachment site of any other oligonucleotide. Typically, such oligonucleotide arrays are “addressable” such that distinct locations are recorded and can be accessed as part of an assay procedure. The knowledge of the location of oligonucleotides on an array make “addressable” arrays useful in hybridization assays. For example, the oligonucleotide probes can be used in an oligonucleotide chip such as those marketed by Affymetrix and described in U.S. Pat. No. 5,143,854; PCT publications WO 90/15070 and 92/10092, the disclosures of which are incorporated herein by reference. These arrays can be produced using mechanical synthesis methods or light directed synthesis methods which incorporate a combination of photolithographic methods and solid phase oligonucleotide synthesis.

The immobilization of arrays of oligonucleotides on solid supports has been rendered possible by the development of a technology generally referred to as “Very Large Scale Immobilized Polymer Synthesis” in which probes are immobilized in a high density array on a solid surface of a chip (see, e.g., U.S. Pat. Nos. 5,143,854; and 5,412,087 and in PCT Publications WO 90/15070, WO 92/10092 and WO 95/11995, each of which are incorporated herein by reference), which describe methods for forming oligonucleotide arrays through techniques such as light-directed synthesis techniques.

In another embodiment, one or more additional polymorphic genes may be analyzed. For example, in addition to the Sp4 and CACNG2 gene polymorphisms useful in the invention, other genes including NTRK2, GRK3 and IMPA1 and -2, ADRBK2, BNDF, GSK3B, INPP1, MARCKS, and/or NR1I2 gene(s) may be performed.

In another aspect, an array of oligonucleotides complementary to subsequences of the target gene are used to determine the identity of the target, measure its amount, and detect differences between the target and a reference wild-type sequence. In one such design, termed 4L tiled array, a set of four probes (A, C, G, T), typically 15-nucleotide oligomers in length are used. In each set of four probes, the perfect complement will hybridize more strongly than mismatched probes. Consequently, hybridization signals of the 15-mer probe tiled array are perturbed by a single base change in the target sequence resulting in a characteristic loss of signal.

Primers useful in the invention include oligonucleotides comprising sequence that flank the underlined sequence above or that comprise a sequence that contains the underlined nucleotide at the 3′ end, thus, for example, preventing primer extension in PCR reactions thus providing a detectable event.

The invention further contemplates, antibodies capable of specifically binding to a variant polypeptide (e.g., a variant Sp4 or CACNG2 polypeptide) encoded in proper frame, based upon transcriptional and translational starts, of the above-identified oligonucleotide sequences (e.g., SEQ ID NOs: 1-38). The invention thus includes isolated, purified, and recombinant polypeptides comprising a contiguous span of at least 4 amino acids, typically at least 6, more commonly at least 8 to 10 amino acids encoded by SEQ ID NOs:1-37 or 38. The contiguous stretch of amino acids comprises the site of a variation (e.g., encoded by a codon comprising the underlined nucleotide above).

In one embodiment of the invention, polynucleotides (e.g., DNA) are obtained from a subject's blood or saliva (or other sample useful for obtaining a polynucleotide sample). The subject's sample is then used to test for a polymorphism (e.g., rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 and/or rs1387923), wherein the presence of one or more of the polymorphism is indicative of a mental disorder. In another aspect, where a mental disorder is identified the subject's response to lithium can then be assayed by measuring a SNP in an NTRK2 gene and/or a GRK3 gene (e.g., rs133845, rs11913984, rs1187287, rs1387923, rs1565445, rs1187352, rs971363 and rs915).

In one aspect, the invention measures a variant in an Sp4 and/or CACNG2 gene if a subject presents with a mood or mental disorder. If a polymorphism a set forth in Table 1 above is identified in an Sp4 and/or CACNG2 gene, the subject is predisposed or diagnosed with a mental disorder.

Methods for diagnostic tests are well known in the art. Generally, the diagnostic test of the invention involves determining whether an individual has a variance or variant form of a gene that is involved in the action of the drug (e.g., lithium) or other treatment or effects of such treatment. Such a variance or variant form of the gene are identified herein and have been identified within the population and are known to be present at a certain frequency. In an exemplary method, the diagnostic test involves amplifying a segment of DNA or RNA (generally after converting the RNA to cDNA) spanning one or more polymorphic regions in a gene (e.g., see Table 1 above). In many cases, the diagnostic test is performed by amplifying a segment of DNA or RNA (cDNA) spanning a polymorphism (e.g., Sp4 and/or CACNG2), or even spanning more than one polymorphisms in the gene (e.g., a CACNG2 region 1 and region 2 polymorphism).

Diagnostic tests useful for practicing the invention typically belong to two types: genotyping tests and haplotyping tests. A genotyping test simply provides the status of a variance or variances in a subject. For example suppose nucleotide 150 of hypothetical gene X on an autosomal chromosome is an adenine (A) or a guanine (G) base. The possible genotypes in any individual are AA, AG or GG at nucleotide 150 of gene X.

In a haplotyping test there is at least one additional variance in gene X, say at nucleotide 810, which varies in the population as cytosine (C) or thymine (T). Thus a particular copy of gene X may have any of the following combinations of nucleotides at positions 150 and 810: 150A-810C, 150A-810T, 150G-810C or 150G-810T. Each of the four possibilities is a unique haplotype. If the two nucleotides interact in either RNA or protein, then knowing the haplotype can be important. The point of a haplotyping test is to determine the haplotypes present in a DNA or cDNA sample (e.g. from a subject).

Based on the identification of variances or variant forms of a gene, a diagnostic test utilizing methods known in the art can be used to determine whether a particular form of the gene, containing specific variances or haplotypes, or combinations of variances and haplotypes, is present in at least one copy, or more than one copy, in a subject. Such tests are performed using DNA or RNA samples collected from blood, cells, tissue scrapings or other cellular materials, and can be performed by a variety of methods including, but not limited to, hybridization with allele-specific probes, enzymatic mutation detection, chemical cleavage of mismatches, mass spectrometry or DNA sequencing, including minisequencing. Diagnostic tests may involve a panel of variances from one or more genes, often on a solid support, which enables the simultaneous determination of more than one variance in one or more genes.

The invention provides polymorphic sequences identified above that may be determined using diagnostic tests. As described herein, such a variance-based diagnostic test can be used to determine whether or not a subject has or is predisposed to having a mental disorder.

Cloning and sequencing of the CACNG2 or Sp4 genes can serve to detect variants useful in predicting a lithium response. Commonly used sequencing techniques can be carried out with commercially available automated sequencers, for example, utilizing fluorescently labeled primers. Other methods of sequence are known in the art.

The target region(s) containing the polymorphism of interest may be amplified using any oligonucleotide-directed amplification method including, but not limited to, polymerase chain reaction (PCR) (U.S. Pat. No. 4,965,188), ligase chain reaction (LCR) (Barany et al., Proc. Natl. Acad. Sci. USA 88:189-93 (1991); WO 90/01069), and oligonucleotide ligation assay (OLA) (Landegren et al., Science 241:1077-80 (1988)). Other known nucleic acid amplification procedures may be used to amplify the target region(s) including transcription-based amplification systems (U.S. Pat. No. 5,130,238; European Patent No. EP 329,822; U.S. Pat. No. 5,169,766; WO 89/06700) and isothermal methods (Walker et al., Proc. Natl. Acad. Sci. USA 89:392-6 (1992)). PCR amplification methods can use primers based upon the sequence identified in Table 1. In one aspect, the primer is designed to have at its 3′ end the single nucleotide polymorphism. Where the polymorphic nucleotide is at the 3′ end, amplification will be inhibited due to inability to extend the primer because of lack of complementarity where a difference exists between the target and the primer.

Ligase Chain Reaction (LCR) techniques can be used. LCR occurs only when the oligonucleotides are correctly base-paired. The Ligase Chain Reaction (LCR), which utilizes the thermostable Taq ligase for ligation amplification, is useful for interrogating loci of a gene (e.g., comprising SEQ ID NO:1-37 or 38). A method of DNA amplification similar to PCR, LCR differs from PCR because it amplifies the probe molecule rather than producing amplicon through polymerization of nucleotides. Two probes are used per each DNA strand and are ligated together to form a single probe. LCR uses both a DNA polymerase enzyme and a DNA ligase enzyme to drive the reaction. Like PCR, LCR requires a thermal cycler to drive the reaction and each cycle results in a doubling of the target nucleic acid molecule. LCR can have greater specificity than PCR. The elevated reaction temperatures permits the ligation reaction to be conducted with high stringency. Where a mismatch occurs, ligation cannot be accomplished. For example, a primer is synthesized in two fragments and annealed to the template with possible mutation at the boundary of the two primer fragments (i.e., the underlined nucleotide above would be found at the 5′ or 3′ end of the oligonucleotide). A ligase ligates the two primers if they match exactly to the template sequence.

In one embodiment, two hybridization probes are designed each with a target specific portion. The first hybridization probe is designed to be substantially complementary to a first target domain of a target polynucleotide (e.g., a polynucleotide fragment) and the second hybridization probe is substantially complementary to a second target domain of a target polynucleotide (e.g., a polynucleotide fragment). In general, each target specific sequence of a hybridization probe is at least about 5 nucleotides long, with sequences of about 15 to 30 being typical and 20 being especially common. In one embodiment, the first and second target domains are directly adjacent, e.g., they have no intervening nucleotides. In this embodiment, at least a first hybridization probe is hybridized to the first target domain and a second hybridization probe is hybridized to the second target domain. If perfect complementarity exists at the junction, a ligation structure is formed such that the two probes can be ligated together to form a ligated probe. If this complementarity does not exist (due to mismatch based upon a variant), no ligation structure is formed and the probes are not ligated together to an appreciable degree. This may be done using heat cycling, to allow the ligated probe to be denatured off the target polynucleotide such that it may serve as a template for further reactions. The method may also be done using three hybridization probes or hybridization probes that are separated by one or more nucleotides, if dNTPs and a polymerase are added (this is sometimes referred to as “Genetic Bit” analysis).

Analysis of point mutations in DNA can also be carried out by using the polymerase chain reaction (PCR) and variations thereof. Mismatches can be detected by competitive oligonucleotide priming under hybridization conditions where binding of the perfectly matched primer is favored. In the amplification refractory mutation system technique (ARMS), primers are designed to have perfect matches or mismatches with target sequences either internal or at the 3′ residue (Newton et al., Nucl. Acids. Res. 17:2503-2516 (1989)). Under appropriate conditions, only the perfectly annealed oligonucleotide functions as a primer for the PCR reaction, thus providing a method of discrimination between normal and variant sequences.

Single nucleotide primer-guided extension assays can also be used, where the specific incorporation of the correct base is provided by the fidelity of a DNA polymerase. Detecting the nucleotide or nucleotide pair at a PS of interest may also be determined using a mismatch detection technique including, but not limited to, the RNase protection method using riboprobes (Winter et al., Proc. Natl. Acad. Sci. USA 82:7575 (1985); Meyers et al., Science 230:1242 (1985)) and proteins which recognize nucleotide mismatches, such as the E. coli mutS protein (Modrich, Ann. Rev. Genet. 25:229-53 (1991)). Alternatively, variant alleles can be identified by single strand conformation polymorphism (SSCP) analysis (Orita et al., Genomics 5:874-9 (1989); Humphries et al., in MOLECULAR DIAGNOSIS OF GENETIC DISEASES, Elles, ed., pp. 321-340, 1996) or denaturing gradient gel electrophoresis (DGGE) (Wartell et al., Nucl. Acids Res. 18:2699-706 (1990); Sheffield et al., Proc. Natl. Acad. Sci. USA 86:232-6 (1989)).

Hybridization techniques can also be used to identify the polymorphisms of the invention and thereby determine a predictive response to lithium. In this aspect, polymorphism(s) are identified based upon the higher thermal stability of a perfectly matched probe compared to the mismatched probe. The hybridization reactions may be carried out in a solid support (e.g., membrane) format, in which the target nucleic acids are immobilized on nitrocellulose or nylon membranes and probed with oligonucleotide probes of the invention. Any of the known hybridization formats may be used, including Southern blots, slot blots, “reverse” dot blots, solution hybridization, solid support based sandwich hybridization, bead-based, silicon chip-based and microtiter well-based hybridization formats.

In one method of diagnosing a susceptibility to psychiatric disorders, hybridization methods, such as Southern analysis, Northern analysis, or in situ hybridizations, can be used (see Current Protocols in Molecular Biology, Ausubel, F. et al., eds., John Wiley & Sons, including all supplements). For example, a biological sample from a test subject (a “test sample”) of genomic DNA, RNA, or cDNA, is obtained from an subject suspected of having, being susceptible to or predisposed for, or carrying a defect for, psychiatric disorders (the “test subject”). The subject can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in an SP4 and/or CACNG2 is present, and/or to determine whether a splicing variant(s) is encoded by SP4 or CACNG2 is present. The presence of the polymorphism or splicing variant(s) can be indicated by hybridization of the gene in the genomic DNA, RNA, or cDNA to a oligonucleotide probe.

In one aspect, a sandwich hybridization assay comprises separating the variant and wild-type target nucleic acids in a sample using a common capture oligonucleotide immobilized on a solid support and then contact with specific probes useful for detecting the variant and wild-type nucleic acids. The oligonucleotide probes are typically tagged with a detectable label.

A polymorphism in a target region of a gene may be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one PS may be detected at once using a set of allele-specific oligonucleotides or oligonucleotide pairs. Typically, the members of the set have melting temperatures within 5° C., and more typically within 2° C., of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The detectable label may be a radioactive label or may be a luminescent, fluorescent of enzyme label. Indirect detection processes typically comprise probes covalently labeled with a hapten or ligand such as digoxigenin (DIG) or biotin. Following the hybridization step, the target-probe duplex is detected by an antibody- or streptavidin-enzyme complex. Enzymes commonly used in DNA diagnostics are horseradish peroxidase and alkaline phosphatase. Direct detection methods include the use of fluorophor-labeled oligonucleotides, lanthanide chelate-labeled oligonucleotides or oligonucleotide-enzyme conjugates. Examples of fluorophor labels are fluorescein, rhodamine and phthalocyanine dyes.

Label detection will be based upon the type of label used in the particular assay. Such detection methods are known in the art. For example, radioisotope detection can be performed by autoradiography, scintillation counting or phosphor imaging. For hapten or biotin labels, detection is with an antibody or streptavidin bound to a reporter enzyme such as horseradish peroxidase or alkaline phosphatase, which is then detected by enzymatic means. For fluorophor or lanthanide-chelate labels, fluorescent signals may be measured with spectrofluorimeters with or without time-resolved mode or using automated microtitre plate readers. With enzyme labels, detection is by color or dye deposition (p-nitropheny phosphate or 5-bromo-4-chloro-3-indolyl phosphate/nitroblue tetrazolium for alkaline phosphatase and 3,3′-diaminobenzidine-NiCl₂ for horseradish peroxidase), fluorescence (e.g., 4-methyl umbelliferyl phosphate for alkaline phosphatase) or chemiluminescence (the alkaline phosphatase dioxetane substrates LumiPhos 530 from Lumigen Inc., Detroit Mich. or AMPPD and CSPD from Tropix, Inc.). Chemiluminescent detection may be carried out with X-ray or polaroid film or by using single photon counting luminometers.

In one aspect, the invention provides oligonucleotides and methods to genotype one or more biallelic markers of the invention by performing a microsequencing assay. It will be appreciated any primer having a 3′ end immediately adjacent to a polymorphic nucleotide may be used. Similarly, it will be appreciated that microsequencing analysis may be performed for any biallelic marker or any combination of biallelic markers of the invention. One aspect of the invention is a solid support which includes one or more microsequencing primers for the SNPs listed herein, or fragments comprising at least 8, at least 12, at least 15, or at least 20 consecutive nucleotides of SEQ ID NO:1-37 or 38 and having a 3′ terminus immediately upstream of the polymorphism.

Hybridization assays based on oligonucleotide arrays rely on the differences in hybridization stability of short oligonucleotides to perfectly matched and mismatched target variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime or smaller. Such a chip may comprise oligonucleotides representative of both the wild-type and variant sequences

Oligonucleotides of the invention can be designed to specifically hybridize to a target region of a polynucleotide containing a desired locus. As used herein, specific hybridization means the oligonucleotide forms an anti-parallel double-stranded structure with the target region under certain hybridizing conditions, while failing to form such a structure when incubated with another region in the polynucleotide or with a polynucleotide lacking the desired locus under the same hybridizing conditions. Typically, the oligonucleotide specifically hybridizes to the target region under conventional high stringency conditions.

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. A nucleic acid molecule is “substantially complementary” to another molecule if it hybridizes to that molecule with sufficient stability to remain in a duplex form under conventional low-stringency conditions. Conventional hybridization conditions are described, for example, in Sambrook et al., Molecular Cloning, A Laboratory Manual, 2nd ed., Cold Spring Harbor Press, Cold Spring Harbor, N.Y. (1989), and in Haymes et al., Nucleic Acid Hybridization, A Practical Approach, IRL Press, Washington, D.C. (1985). While perfectly complementary oligonucleotides are used in most assays for detecting polymorphisms, departures from complete complementarity are contemplated where such departures do not prevent the molecule from specifically hybridizing to the target region. For example, an oligonucleotide primer may have a non-complementary fragment at its 5′ or 3′ end, with the remainder of the primer being complementary to the target region. Those of skill in the art are familiar with parameters that affect hybridization; such as temperature, probe or primer length and composition, buffer composition and salt concentration and can readily adjust these parameters to achieve specific hybridization of a nucleic acid to a target sequence.

A variety of hybridization conditions may be used in the disclosure, including high, moderate and low stringency conditions; see for example Maniatis et al., Molecular Cloning: A Laboratory Manual, 2d Edition, 1989, and Short Protocols in Molecular Biology, ed. Ausubel, et al., hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the polyadenylated mRNA target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of helix destabilizing agents such as formamide. The hybridization conditions may also vary when a non-ionic backbone, i.e., PNA is used, as is known in the art. In addition, cross-linking agents may be added after target binding to cross-link, i.e., covalently attach, the two strands of the hybridization complex.

Using the methods described herein, the genotype or haplotype for an Sp4 and/or CACNG2 gene of a subject may be determined by hybridization of a nucleic acid sample containing one or both copies of the gene, mRNA, cDNA or fragment(s) thereof, to nucleic acid arrays and subarrays such as described in WO 95/11995. The arrays would contain a battery of allele-specific oligonucleotides representing each of the PSs to be included in the genotype or haplotype.

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, the disclosure of which is incorporated herein by reference in its entirety, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip. For genotyping biallelic markers, the microfluidic system may integrate nucleic acid amplification, microsequencing, capillary electrophoresis and a detection method such as laser-induced fluorescence detection.

The invention also contemplates the use of immunoassay techniques for measurement of the polymorphisms identified herein for determining the probability or diagnosis of a mental disorder. For example, point mutations (as identified in SEQ ID NO:1-38) may alter the structure of the proteins for which these gene encode. These altered polypeptides (e.g., CACNG2 or Sp4 polypeptides) can be isolated and used to prepare antisera and monoclonal antibodies that specifically detect the mutated gene products and not those of non-mutated or wild-type gene products. Mutated gene products also can be used to immunize animals for the production of polyclonal antibodies. Recombinantly produced peptides can also be used to generate antibodies. For example, a recombinantly produced fragment of a variant polypeptide can be injected into a mouse along with an adjuvant so as to generate an immune response. Murine immunoglobulins which bind the recombinant fragment with a binding affinity of at least 1×10⁷ M⁻¹ can be harvested from the immunized mouse as an antiserum, and may be further purified by affinity chromatography or other means. Additionally, spleen cells are harvested from the mouse and fused to myeloma cells to produce a bank of antibody-secreting hybridoma cells. The bank of hybridomas can be screened for clones that secrete immunoglobulins which bind the recombinantly produced fragment with an affinity of at least 1×10⁶ M⁻¹. More specifically, immunoglobulins that selectively bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Polynucleotides capable of expressing the desired variant polypeptides can be generated using techniques skilled in the art based upon the identified polymorphisms herein. Such polynucleotides can be expressed in hosts, wherein the polynucleotide is operably linked to (i.e., positioned to ensure the functioning of) an expression control sequence. Expression vectors are typically replicable in the host organisms either as episomes or as an integral part of the host chromosome. Expression vectors can contain selection markers (e.g., markers based on tetracyclin resistance or hygromycin resistance) to permit detection and/or selection of those cells transformed with the desired polynucleotide.

Polynucleotides encoding a variant polypeptide may include sequences that facilitate transcription and translation of the coding sequences such that the encoded polypeptide product is produced. Construction of such polynucleotides is known in the art. For example, such polynucleotides can include a promoter, a transcription termination site (polyadenylation site in eukaryotic expression hosts), a ribosome binding site, and, optionally, an enhancer for use in eukaryotic expression hosts, and, optionally, sequences necessary for replication of a vector.

Prokaryotes can be used as host cells for the expression of a variant polypeptides, such techniques are known in the art. Other microbes, such as yeast, may also be used for expression. In addition to microorganisms, mammalian tissue cell culture may also be used to express and produce polypeptides of the invention. Eukaryotic cells useful in the methods of the invention include the CHO cell lines, various COS cell lines, HeLa cells, myeloma cell lines, Jurkat cells, and so forth. Expression vectors for these cells can include expression control sequences, such as an origin of replication, a promoter, an enhancer, an necessary information processing sites, such as ribosome binding sites, RNA splice sites, polyadenylation sites, and transcriptional terminator sequences.

Linkage disequilibrium is the non-random association of alleles at two or more loci (e.g., CACNG2 region-1 and -2) and represents a powerful tool for mapping genes involved in disease traits. Biallelic markers, because they are densely spaced in the human genome and can be genotyped in more numerous numbers than other types of genetic markers, are particularly useful in genetic analysis based on linkage disequilibrium.

Briefly, when a mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombinations occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the locus. Consequently, the amount of linkage disequilibrium between an allele and closely linked genetic markers may yield valuable information regarding the location of a disease gene. For fine-scale mapping of a disease locus, it is useful to have some knowledge of the patterns of linkage disequilibrium that exist between markers in the studied region.

A number of methods can be used to calculate linkage disequilibrium between any two genetic positions, in practice linkage disequilibrium is measured by applying a statistical association test to haplotype data taken from a population.

While direct haplotyping of both copies of the gene can be performed with each copy of the gene analyzed independently, it is also envisioned that direct haplotyping could be performed simultaneously if the two copies are labeled with different tags, or are otherwise separately distinguishable or identifiable. For example, if first and second copies of the gene are labeled with different first and second fluorescent dyes, respectively, and an allele-specific oligonucleotide labeled with yet a third different fluorescent dye is used to assay the polymorphism(s), then detecting a combination of the first and third dyes would identify the polymorphism in the first gene copy while detecting a combination of the second and third dyes would identify the polymorphism in the second gene copy.

In both the direct and indirect haplotyping methods, the identity of a nucleotide (or nucleotide pair) at a PS(s) in the amplified target region may be determined by sequencing the amplified region(s) using conventional methods. If both copies of the gene are represented in the amplified target, it will be readily appreciated by the skilled artisan that only one nucleotide will be detected at a PS in individuals who are homozygous at that site, while two different nucleotides will be detected if the individual is heterozygous for that site. The polymorphism may be identified directly, known as positive-type identification, or by inference, referred to as negative-type identification. For example, where a polymorphism is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

Once a first biallelic marker has been identified in a genomic region of interest, the practitioner of ordinary skill in the art, using the teachings of the invention, can easily identify additional biallelic markers in linkage disequilibrium with this first marker. As mentioned before, any marker in linkage disequilibrium with a first marker associated with a trait will be associated with the trait. Therefore, once an association has been demonstrated between a given biallelic marker and a trait, the discovery of additional biallelic markers associated with this trait is of interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

The invention provides assays which may be used to identify subjects that respond to a particular therapeutic treatment. For example, in one aspect, the assays of the invention identify one or more SNPs that have been associated with a clinical manifestation of a mental disorder. Thus, identifying such SNPs in a subject can provide information indicative of that subject's predisposition for, or presence of, a mental disorder.

The invention also provides cell and animal models, including primate and mouse, of mental disorders. In one aspect, provided are non-cell based, cell based and animal based assays for the identification of compounds that affect CACNG2 or Sp4 expression or activity. The invention has identified polymorphisms CACNG2 and Sp4 associated with mental disorders. Accordingly, genetically engineered organisms (including non-human transgenic animals) comprising a homolog or variant comprising a SNP of Table 1 can be used to assess responsiveness of the organism to compounds useful in treating mental disorders.

The invention also relates to an assay for identifying agents which alter the expression of a mutant Sp4 or CACNG2 (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein. For example, a solution containing a nucleic acid encoding a mutant Sp4 or CACNG2 polypeptide can be contacted with an agent to be tested. The solution can comprise, for example, cells containing the nucleic acid or cell lysate containing the nucleic acid; alternatively, the solution can be another solution which comprises elements necessary for transcription/translation of the nucleic acid. Cells not suspended in solution can also be employed, if desired. The level and/or pattern of Sp4 or CACNG2 expression (e.g., the level and/or pattern of mRNA or of protein expressed, such as the level and/or pattern of different splicing variants) is assessed, and is compared with the level and/or pattern of expression in a control (i.e., the level and/or pattern of the Sp4 or CACNG2 expression in the absence of the agent to be tested). If the level and/or pattern in the presence of the agent differs, by an amount or in a manner that is statistically significant, from the level and/or pattern in the absence of the agent, then the agent is an agent that alters the expression of mutant Sp4 or CACNG2. Enhancement of mutant SP4 expression indicates that the agent is an agonist of mutant SP4 activity. Similarly, inhibition of mutant Sp4 or CACNG2 expression indicates that the agent is an antagonist of mutant Sp4 or CACNG2 activity. In another embodiment, the level and/or pattern of Sp4 or CACNG2 polypeptide(s) (e.g., different splicing variants) in the presence of the agent to be tested, is compared with a control level and/or pattern that has previously been established. A level and/or pattern in the presence of the agent that differs from the control level and/or pattern by an amount or in a manner that is statistically significant indicates that the agent alters SP4 expression.

In one aspect, cell based assays using recombinant or non-recombinant cells may be used to identify compounds which modulate CACNG2 or Sp4 expression or activity. A cell based assay of the invention encompasses a method for identifying a test compound for the treatment of mental disorders (e.g., schizophrenia or bipolar disorder) comprising (a) exposing a cell comprising a mutant allele as set forth in Table 1 to a test compound at a concentration and time sufficient to ameliorate an endpoint related to a mental disorder (e.g., schizophrenia or bipolar disorder), and (b) determining the level of CACNG2 or Sp4 activity in a cell. Such measurement can include measuring, for example, transcription level, polypeptide expression, localization or activity. Typically, the test compound is a compound capable of or suspected to be capable of ameliorating a symptom of, for example, schizophrenia, bipolar disorder or a related disorder. For example, if the level of the activity in the presence of the agent differs, by an amount that is statistically significant, from the level of the activity in the absence of the agent, then the agent is an agent that alters the activity of an Sp4 or CACNG2 polypeptide. An increase in the level of Sp4 or CACNG2 polypeptide activity relative to a control, indicates that the agent is an agent that enhances (is an agonist of) Sp4 or CACNG2 activity. Similarly, a decrease in the level of Sp4 or CACNG2 polypeptide activity relative to a control, indicates that the agent is an agent that inhibits (is an antagonist of) Sp4 or CACNG2 activity. In another embodiment, the level of activity of an Sp4 or CACNG2 polypeptide or derivative or fragment thereof in the presence of the agent to be tested, is compared with a control level that has previously been established. A level of the activity in the presence of the agent that differs from the control level by an amount that is statistically significant indicates that the agent alters Sp4 or CACNG2 activity.

In another embodiment, a CACNG2 or Sp4 polynucleotide, or fragments thereof, is cloned into expression vectors. The polynucleotide or fragment is then expressed the expression product purified by size, charge, immunochromatography or other techniques familiar to those skilled in the art. Following purification, the expression product(s) are labeled using techniques known to those skilled in the art. The labeled proteins are incubated with cells or cell lines derived from a variety of organs or tissues to allow the proteins to bind to any receptor present on the cell surface. Following the incubation, the cells are washed to remove non-specifically bound protein. The labeled proteins are detected. A test compound binding may be analyzed by conducting a competition analysis in which various amounts of a test compound are incubated along with the labeled protein. The amount of labeled protein bound to the cell surface decreases as the amount of competitive unlabeled test compound increases.

In more than one embodiment of the above assay methods of the invention, it may be desirable to immobilize a Sp4 and/or CACNG2 polypeptide (e.g., a mutant Sp4 and/or CACNG2), the Sp4 and/or CACNG2 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows Sp4 and/or CACNG2 polypeptide or an Sp4 and/or CACNG2 binding agent to be bound to a matrix or other solid support.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent (e.g., based on both biological measurements and behavioral analysis).

Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a polypeptide encoded by SP4 (e.g., a mutant SP4), or to alter expression of SP4, by contacting the polypeptide or the gene (or contacting a cell comprising the polypeptide or the gene) with the agent identified as described herein.

An animal-based assays may also be used to identify compounds which modulate Sp4 and/or CACNG2 activity. Thus, the invention comprises treating an animal comprising a homolog or variant comprising SEQ ID NO:1-37 and/or 38 with a test compound. In one aspect, an animal-based assay of the invention encompasses a method for identifying a test compound for the treatment of mood disorder comprising (a) exposing an animal to a test compound at a concentration and time sufficient to ameliorate an endpoint related to a mood disorder (e.g., prepulse inhibition), and (b) determining the effect of the test compound on the endpoint and/or the level of Sp4 and/or CACNG2 activity in the animal. In one aspect, the animal is a primate, a non-human transgenic animal (e.g., a primate or a mouse).

Any suitable test compound may be used with the screening methods of the invention. Examples of compounds that may be screened by the methods of the invention include small organic or inorganic molecules, nucleic acids (e.g., ribozymes, antisense molecules), including polynucleotides from random and directed polynucleotide libraries, peptides, including peptides derived from random and directed peptide libraries, soluble peptides, fusion peptides, and phosphopeptides, antibodies including polyclonal, monoclonal, chimeric, humanized, and anti-idiotypic antibodies, and single chain antibodies, FAb, F(ab′)₂ and FAb expression library fragments, and epitope-binding fragments thereof. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des., 12:145). In certain aspects, a compound capable of ameliorating or exacerbating a symptom or endpoint of schizophrenia, bipolar disorder or a related disorder may include, by way of example, antipsychotic drugs in general, neuroleptics, atypical neuroleptics, antidepressants, anti-anxiety drugs, noradrenergic agonists and antagonists, dopaminergic agonists and antagonists, serotonin reuptake inhibitors, benzodiazepines.

In further methods, peptides, drugs, fatty acids, lipoproteins, or small molecules which interact with the Sp4 and/or CACNG2 protein, or a fragment comprising a contiguous span of at least 4 amino acids, at least 6 amino acids, or typically at least 8 to 10 amino acids or more of sequences corresponding to the SNPs of SEQ ID NO:1-37 and/or 38. The molecule to be tested for binding is labeled with a detectable label, such as a fluorescent, radioactive, or enzymatic tag and placed in contact with immobilized Sp4 and/or CACNG2 protein or a variant thereof under conditions which permit specific binding to occur. After removal of non-specifically bound molecules, bound molecules are detected using appropriate means.

In one embodiment, the invention provides a kit useful for identifying polymorphisms associated or predictive of lithium response. For example, the kit of the invention can comprise one or more oligonucleotides designed for identifying both alleles at each PS in the set of one or more PSs. In another embodiment, the kit further comprises a manual with instructions for (a) performing one or more reactions on a human nucleic acid sample to identify the allele or alleles present in the subject at each PS in the set of one or more PSs.

The oligonucleotides in a kit of the invention may also be immobilized on or synthesized on a solid surface such as a microchip, bead, or glass slide (see, e.g., WO 98/20020 and WO 98/20019). Such immobilized oligonucleotides may be used in a variety of polymorphism detection assays, including but not limited to probe hybridization and polymerase extension assays. Immobilized oligonucleotides useful in practicing the invention may comprise an ordered array of oligonucleotides designed to rapidly screen a nucleic acid sample for polymorphisms in multiple genes at the same time.

Kits of the invention may also contain other components such as hybridization buffer (e.g., where the oligonucleotides are to be used as allele-specific probes) or dideoxynucleotide triphosphates (ddNTPs; e.g., where the alleles at the polymorphic sites are to be detected by primer extension). In a one embodiment, the set of oligonucleotides consists of primer-extension oligonucleotides. The kit may also contain a polymerase and a reaction buffer optimized for primer-extension mediated by the polymerase. Preferred kits may also include detection reagents, such as biotin- or fluorescent-tagged oligonucleotides or ddNTPs and/or an enzyme-labeled antibody and one or more substrates that generate a detectable signal when acted on by the enzyme. It will be understood by the skilled artisan that the set of oligonucleotides and reagents for performing the genotyping or haplotyping assay will be provided in separate receptacles placed in the container if appropriate to preserve biological or chemical activity and enable proper use in the assay.

It is also contemplated that the above described methods and compositions of the invention may be utilized in combination with identifying genotype(s) and/or haplotype(s) for other genomic regions.

Nucleic acid samples, for example for use in variance identification, can be obtained from a variety of sources as known to those skilled in the art, or can be obtained from genomic or cDNA sources by known methods.

The invention also pertains to pharmaceutical compositions comprising polynucleotides (e.g., wild type Sp4 and/or CACNG2) described herein; comprising polypeptides described herein; comprising an Sp4 and/or CACNG2 therapeutic agent; and/or comprising an agent that alters (e.g., enhances or inhibits) Sp4 and/or CACNG2 expression or Sp4 and/or CACNG2 polypeptide activity.

Suitable pharmaceutically acceptable carriers include, but are not limited to, water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, and the like.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, and the like.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of psychiatric disorders, and should be decided according to the judgment of a practitioner and each patient's circumstances. Effective doses may be extrapolated from dose-response curves derived from in vitro or animal model test systems.

The invention also pertains to methods of treatment (prophylactic and/or therapeutic) for mental disorders, using an Sp4 and/or CACNG2 therapeutic agent. An “Sp4 and/or CACNG2 therapeutic agent” is an agent, used for the treatment of mental disorders, that alters (e.g., enhances or inhibits) Sp4 and/or CACNG2 polypeptide activity and/or Sp4 and/or CACNG2 gene expression, as described herein. Sp4 and/or CACNG2 therapeutic agents can alter Sp4 and/or CACNG2 polypeptide activity or gene expression by a variety of means, such as, for example, by providing additional Sp4 and/or CACNG2 polypeptide or by upregulating the transcription or translation of Sp4 and/or CACNG2; by altering post translational processing of the Sp4 and/or CACNG2 polypeptide; by altering transcription of Sp4 and/or CACNG2 splicing variants; by interfering with Sp4 and/or CACNG2 polypeptide activity (e.g., by binding to an Sp4 and/or CACNG2 polypeptide); by altering the interaction between Sp4 and/or CACNG2 polypeptide and an Sp4 and/or CACNG2 polypeptide binding agent (e.g., a receptor); by altering the activity of an SP4 polypeptide binding agent; or by down regulating the transcription or translation of Sp4 and/or CACNG2. Representative Sp4 or CACNG2 therapeutic agents include the following: nucleic acids or fragments or derivatives thereof described herein, particularly nucleotides encoding a wtSp4 or wtCACNG2 (e.g., a gene, cDNA, and/or mRNA, such as a nucleic acid encoding an Sp4 or CACNG2 polypeptide or active fragment or derivative thereof, or an oligonucleotide; Sp4 or CACNG2 wild-type polypeptides; Sp4 or CACNG2 binding agents; peptidomimetics; fusion proteins or prodrugs thereof; antibodies (e.g., an antibody to a mutant Sp4 or CACNG2 polypeptide, or an antibody to a non-mutant Sp4 or CACNG2 polypeptide, or an antibody to a particular splicing variant encoded by Sp4 or CACNG2); ribozymes; other small molecules; agents that alter interaction between Sp4 or CACNG2 polypeptide and an Sp4 or CACNG2 polypeptide binding agent; agents that alter activity of an Sp4 or CACNG2 polypeptide binding agent (e.g., an agent that alters (e.g., enhances or inhibits) expression and/or activity of an Sp4 or CACNG2 polypeptide binding agent; and other agents that alter (e.g., enhance or inhibit) Sp4 or CACNG2 gene expression or polypeptide activity, that alter post translational processing of the Sp4 or CACNG2 polypeptide, or that regulate transcription of Sp4 or CACNG2 splicing variants.

In one embodiment, the Sp4 or CACNG2 therapeutic agent is a nucleic acid encoding one or more Sp4 or CACNG2 polypeptides; in another embodiment, the Sp4 or CACNG2 therapeutic agent is a nucleic acid comprising a fragment of Sp4 or CACNG2, such as a regulatory region of Sp4 or CACNG2; in yet another embodiment, the Sp4 or CACNG2 therapeutic agent is a nucleic acid comprising the Sp4 or CACNG2 regulatory region and also a nucleic acid encoding one or more Sp4 or CACNG2 polypeptides (or fragments or derivatives thereof).

The term, “treatment” as used herein, refers not only to ameliorating symptoms associated with the disease, but also preventing or delaying the onset of the disease, and also lessening the severity or frequency of symptoms of the disease. The therapy is designed to alter (e.g., inhibit or enhance), replace or supplement activity of an Sp4 or CACNG2 polypeptide in an individual.

For example, an Sp4 or CACNG2 therapeutic agent can be administered in order to up regulate or increase the expression or availability of the Sp4 or CACNG2 gene or of specific splicing variants of Sp4 or CACNG2, or, conversely, to down regulate or decrease the expression or availability of a mutant Sp4 or CACNG2 gene or specific splicing variants of Sp4 or CACNG2. Up regulation or increasing expression or availability of a native Sp4 or CACNG2 or of a particular splicing variant could interfere with or compensate for the expression or activity of a defective gene or another splicing variant; down regulation or decreasing expression or availability of a native Sp4 or CACNG2 or of a particular splicing variant could minimize the expression or activity of a defective gene or the particular splicing variant and thereby minimize the impact of the defective gene or the particular splicing variant.

An Sp4 or CACNG2 polynucleotide or a cDNA encoding the Sp4 or CACNG2 polypeptide, either by itself or included within a vector, can be introduced into cells (either in vitro or in vivo) such that the cells produce native Sp4 or CACNG2 polypeptide. If necessary, cells that have been transformed with the gene or cDNA or a vector comprising the gene or cDNA can be introduced (or re-introduced) into an individual affected with the disease. Thus, cells which, in nature, lack native Sp4 or CACNG2 expression and activity, or have mutant Sp4 or CACNG2 expression and activity, or have expression of a disease-associated Sp4 or CACNG2 can be engineered to express Sp4 or CACNG2 polypeptide or an active fragment of the Sp4 or CACNG2 polypeptide (or a different variant of Sp4 or CACNG2 polypeptide). In one embodiment, nucleic acid encoding the Sp4 or CACNG2 polypeptide, or an active fragment or derivative thereof, can be introduced into an expression vector, such as a viral vector, and the vector can be introduced into appropriate cells in an animal. Other gene transfer systems, including viral and nonviral transfer systems, can be used.

Alternatively, nonviral gene transfer methods, such as calcium phosphate coprecipitation, mechanical techniques (e.g., microinjection); membrane fusion-mediated transfer via liposomes; or direct DNA uptake, can also be used. Alternatively, in another embodiment of the invention, a nucleic acid of the invention; a nucleic acid complementary to a nucleic acid of the invention; or a portion of such a nucleic acid can be used in “antisense” therapy, in which a nucleic acid (e.g., an oligonucleotide) which specifically hybridizes to the mRNA and/or genomic DNA of a mutant Sp4 or CACNG2 is administered or generated in situ. The antisense nucleic acid that specifically hybridizes to the mRNA and/or DNA inhibits expression of the mutant Sp4 or CACNG2 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the invention can be delivered, for example, as an expression plasmid as described above. When the plasmid is transcribed in the cell, it produces RNA which is complementary to a portion of the mRNA and/or DNA which encodes Sp4 or CACNG2 polypeptide.

Alternatively, the antisense construct can be an oligonucleotide probe which is generated ex vivo and introduced into cells; it then inhibits expression by hybridizing with the mRNA and/or genomic DNA of mutant Sp4 or CACNG2.

In one embodiment, the oligonucleotide probes are modified oligonucleotides which are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al. ((1988) Cancer Res 48:2659-2668). With respect to antisense DNA, oligodeoxyribonucleotides derived from the translation initiation site.

To perform antisense therapy, oligonucleotides (mRNA, cDNA or DNA) are designed that are complementary to mRNA encoding a mutant Sp4 or CACNG2. The antisense oligonucleotides bind to Sp4 or CACNG2 mRNA transcripts and prevent translation. Absolute complementarity, although preferred, is not required.

The antisense molecules are delivered to cells which express Sp4 or CACNG2 (e.g., a mutant Sp4 or CACNG2) in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, In one embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous Sp4 or CACNG2 transcripts and thereby prevent translation of the mutant Sp4 or CACNG2 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct which can be introduced directly into the tissue site. Alternatively, viral vectors can be used which selectively infect the desired tissue, in which case administration may be accomplished by another route (e.g., systematically).

Endogenous mutant Sp4 or CACNG2 expression can also be reduced by inactivating or “knocking out” Sp4 or CACNG2 or its promoter using targeted homologous recombination (e.g., see Smithies et al. (1985) Nature 317:230-234; Thomas & Capecchi (1987) Cell 51:503-512; Thompson et al. (1989) Cell 5:313-321). Alternatively, endogenous mutant Sp4 or CACNG2 expression can be reduced by targeting deoxyribonucleotide sequences complementary to the regulatory region of Sp4 or CACNG2 (i.e., the Sp4 or CACNG2 promoter and/or enhancers) to form triple helical structures that prevent transcription of Sp4 or CACNG2 in target cells in the body. (See generally, Helene, C. (1991) Anticancer Drug Des., 6(6):569-84; Helene, C., at al. (1992) Ann, N.Y. Acad. Sci., 660:27-36; and Maher, L. J. (1992) Bioassays 14 (12):807-15).

### EXAMPLES

In a study of 20 bipolar families, genome-wide evidence showed linkage at 22q13. These linkage data supported the presence of two distinct genes for bipolar disorder on 22q. One of these is at the GRK3 gene on 22q12. In this invention, a second gene at 22q13 was identified as CACNG2 also known as Stargazin.

Methods: a two phase study was employed using two independent samples from subjects. In the first phase, 189 SNPs were genotyped in a 5 MB region flanking the linkage peak at D22S278 in a sample of 499 individuals from 156 families. Genotyping was conducted by Illumina. In phase 2, a 1.5 Mb interval was genotyped using 159 SNPs in 1138 subjects. The genotyping in phase 2 was conducting using ABI SNPlex. Subjects were probands and sibs with bipolar I disorder and their parents from families ascertained for linkage studies. They came from two samples: the NIMH Genetics Initiative for Bipolar Disorder waves 1-4, and a collection through the University of California, San Diego. The transmission disequilibrium test (TDT) was employed to test for association between SNPs and bipolar disorder. TDT statistics were calculated using the UNPHASED program for single SNPs and moving window haplotypes of 2 and 3 markers each.

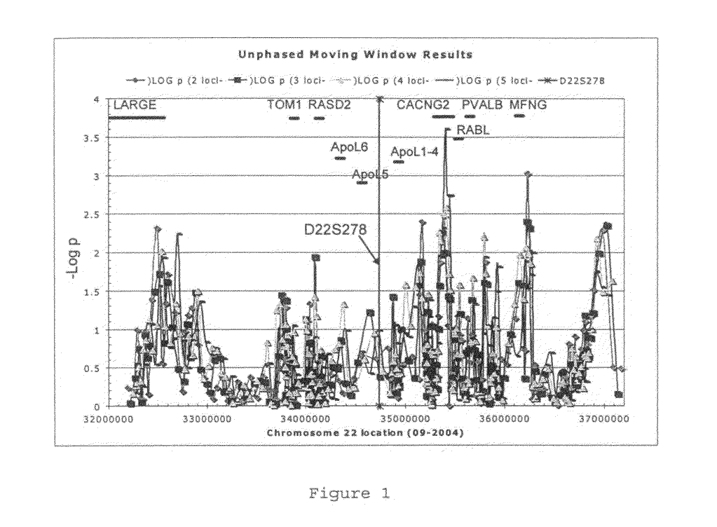

Results: The phase 1 results are shown in FIG. 1. The maximum evidence of association was p=0.005 at rs738518. This SNP is 684 kb telomeric of D22S278 and flanks CACNG2, a calcium channel gamma subunit associated with absence epilepsy in the mouse. A modest linkage disequilibrium was identified with rs140005 (p=0.016), also near CACNG2. These data suggested association of bipolar disorder to the CACNG2 gene. However, these results were not statistically significant given the number of SNPs examined and only modestly greater than other regions.

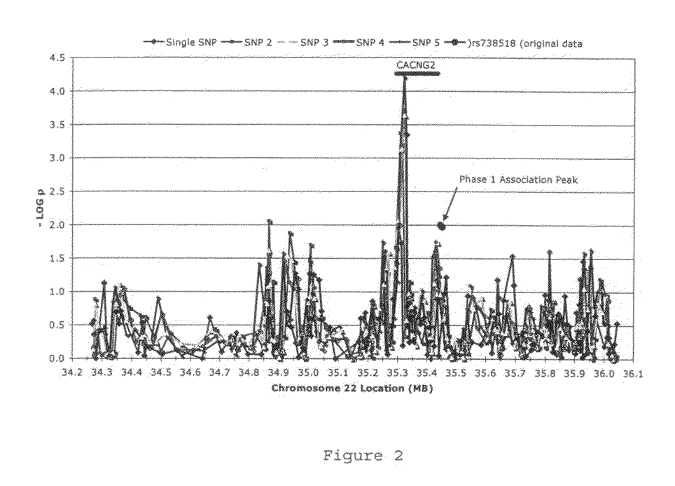

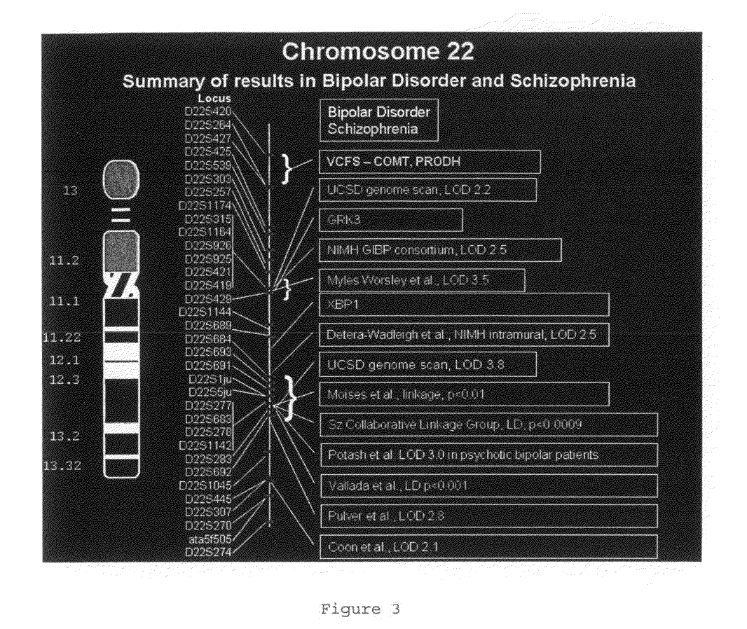

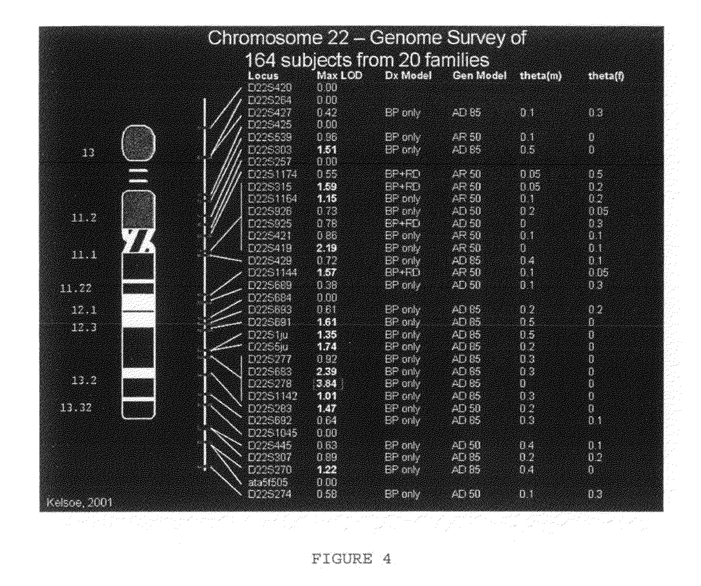

To replicate these findings, an additional 159 SNPs over a 1.78 MB region in 1136 individuals from 283 independent families were examined. These results are illustrated in FIG. 2. Evidence for linkage disequilibrium from the single locus analysis at SNPs rs2267341 (p=0.011) and rs2283981 (p=0.019) was identified, both of which are intronic SNPs of CACNG2. The maximum evidence for linkage disequilibrium using haplotypes was also in this same area, the 2- and 3-marker moving window yielded p=0.0004 and p=0.0002, respectively. The maximum evidence for linkage disequilibrium across the combined family set yielded the best results at the same markers (individual TDT p=0.003, 2-SNP haplotype p=0.0005, 3-SNP haplotype p=0.001).

In both samples of subjects the maximum evidence for association to bipolar disorder was to SNPs and haplotypes in the CACNG2 gene. The results are consistent with CACNG2 as a susceptibility locus for bipolar disorder.

These results have interesting implications for the molecular mechanism of bipolar disorder. CACNG2, also called stargazin, is a membrane associated protein that is involved in the trafficking and association of AMPA and NMDA receptors. CACNG2 may also play a role in modulating the efficiency of signal transduction for AMPA receptors. A mutation in the CACNG2 gene has been shown to result in an absence epilepsy phenotype in the stargazer mouse. NMDA receptors have been implicated in psychosis and the mechanism of action of antipsychotics. The hallucinogen, PCP, acts at NMDA receptors. Atypical antipsychotics may derive some of their efficacy from action at NMDA receptors. Similarly, partial complex seizure disorder has been associated with bipolar disorder and anticonvulsants are used as primary treatment for bipolar disorder.

Material and Methods for Sp4 Analysis. Sp4 null mutant mice, Immunohistochemical analysis, and Stereological analysis were used.

Sp4 null mice demonstrated phenotypic including reduced prepulse inhibition and increased startle response. Reduced expression of Sp4 causes hippocampal vacuolization, astrogliosis as well as down regulation of neurotropin-3 expression. Sp4 hypomorphic mice demonstrated deficits in memory and other behavioral abnormalities. Null mice also demonstrated a reduction in time spent freezing (e.g., a conditioned fear context) and a decreased mean latency to step in passive avoidance tests.

Reactivation of null Sp4 mice by cre-lox recombination with a wild type gene resulting in rescue of mice. Rescued mice did not display behavior abnormalities, absence of vacuolization, and normal expression of neurotrophin-3.

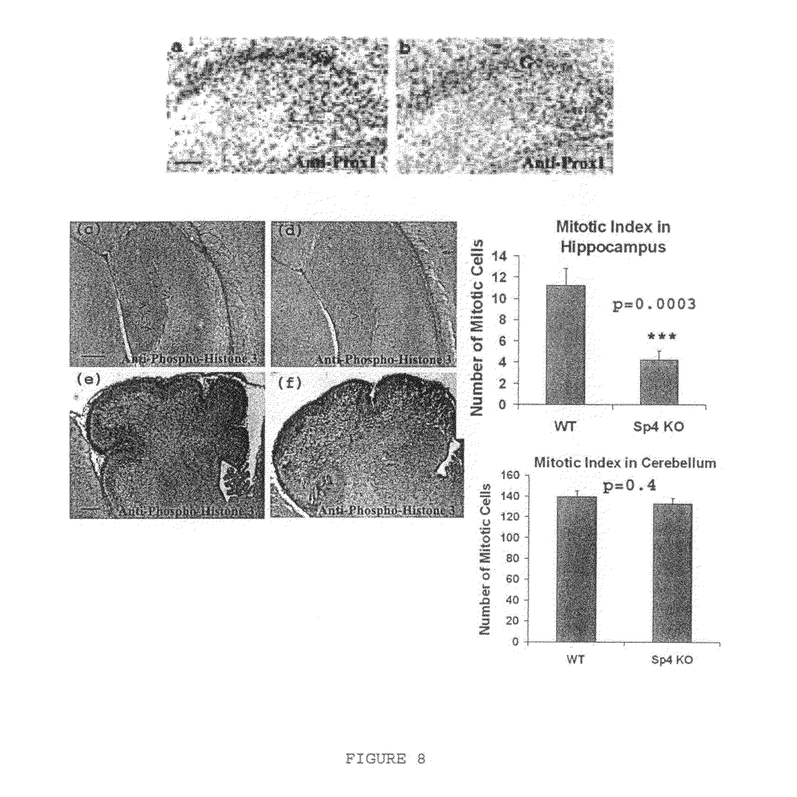

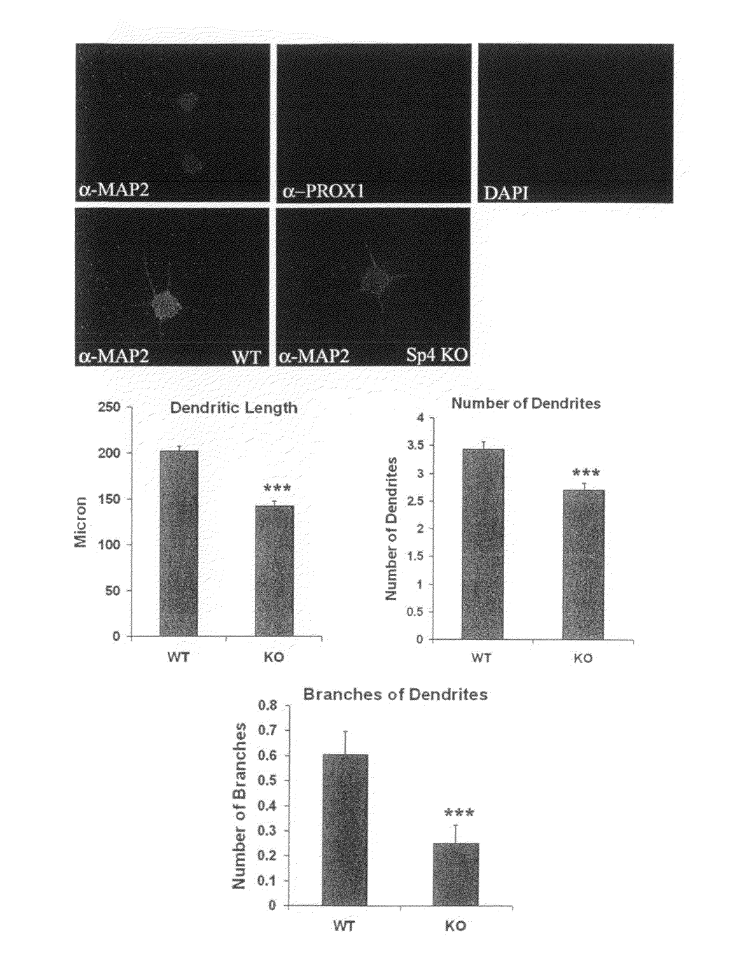

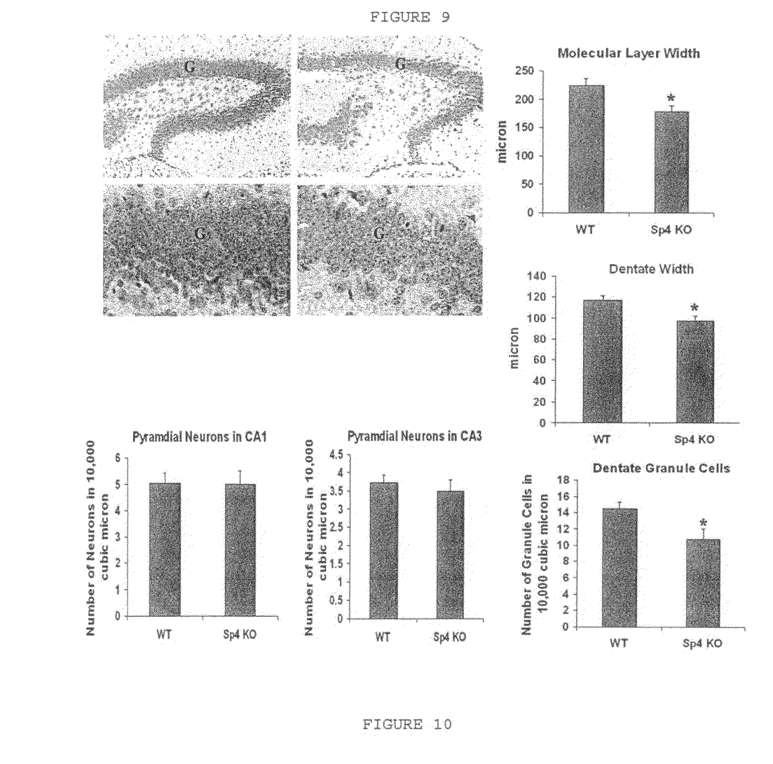

The complete absence of Sp4 gene attenuates neuronal cell proliferation in postnatal hippocampus. The Sp4 knockout mice display reduced size of dentate gyrus associated with both decreased granule cells and abnormal synaptogenesis in the molecular layer (see, e.g., FIGS. 8-10).

Genome-wide linkage analysis identified a susceptibility locus for bipolar disorder on human chromosome 7p15 in the families obtained from the NIMH Genetics Initiative for Bipolar Disorder first-wave pedigree collection. To further examine whether human SP4 is a risk gene for bipolar disorder, association studies were conducted. Most of the samples coming from the NIMH Genetics Initiative for Bipolar Disorder first-, second-, third-, and fourth-wave pedigree collections. Nine SNPs encompassing human Sp4 genomic locus were selected for initial association studies. ETDT was used for association analysis.

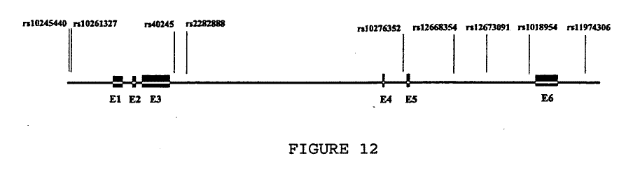

Referring to FIG. 12, Markers 1-9 are shown (from left to right (e.g., Marker 6 is rs12668354). The single SNP Marker 7 was significantly associated with bipolar disorder with a pvalue of 0.0008. Another three-SNP haplotypes also displayed a significant association with bipolar disorder.

| Name | MAF | T:U | Chi Squared | p-value |
|---|---|---|---|---|
| hCV2625401 | 0.234 | 187:187 | 0.0 | 1.0000 |
| (rs10245440) | | | | |
| hCV2625402 | 0.459 | 253:226 | 1.522 | 0.2173 |
| (rs10261327) | | | | |
| rs40245 | 0.378 | 250:267 | 0.559 | 0.4547 |
| rs2282888 | 0.397 | 229:265 | 2.623 | 0.1053 |
| hCV2625432 | 0.439 | 265:292 | 1.309 | 0.2526 |
| (rs10276352) | | | | |
| hCV11828813 | 0.332 | 213:257 | 4.119 | 0.0424 |
| (rs12668354) | | | | |
| hCV2625441 | 0.271 | 251:181 | 11.343 | 0.0008 |
| (rs12673091) | | | | |
| rs1018954 | 0.417 | 277:261 | 0.476 | 0.4902 |
| hCV2625454 | 0.48 | 247:299 | 4.952 | 0.0261 |
| (rs11974306) | | | | |

A number of embodiments have been described. Nevertheless, it will be understood that various modifications may be made without departing from the spirit and scope of the description. Accordingly, other embodiments are within the scope of the following claims.

What is claimed is:

1. A method comprising:
(a) contacting a sample comprising polynucleotides from a subject with at least one probe comprising an oligonucleotide that interacts with a single nucleotide polymorphism (SNP) in CACNG2 and/or in Sp4 polynucleotide; and
(b) detecting the binding of the at least one probe to a polynucleotide in the sample.

2. The method of claim 1, wherein the probe comprises SEQ ID NO:1-37 or 38 of a fragment thereof comprising the SNP.

3. The method of claim 1, wherein the at least one probe comprises at least 8 contiguous nucleotides of SEQ ID NO: 1-37 or 38 and containing nucleotide 11.

4. The method of claim 1, further comprising contacting the sample with at least one additional probe comprising 8 contiguous nucleotides selected from SEQ ID NO:1-37 and 38 and containing nucleotide 11.

5. The method of claim 1, wherein the probe comprises nucleotide 11 of SEQ ID NO:1-37 or 38 at the 5′ or 3′ end.

6. A method for determining whether a subject has a mental or mood disorder, comprising:
contacting a sample with at least one probe comprising at least 8 contiguous nucleotides of SEQ ID NO:1-37 or 38, and containing nucleotide 11 or the complement thereof; and
determining if the sample comprises a polynucleotide molecule that hybridizes to the probe.

7. The method of claim 6, further comprising contacting the sample with at least one additional probe comprising 8 contiguous nucleotides selected from SEQ ID NO: 1-37 or 38 and containing nucleotide 11.

8. The method of claim 1 or 6, further comprising diagnosing a mental disorder or clinical symptom in the subject selected from the group consisting of euphoric mania, dysphoric mania, Bipolar I, Rapid Cycling, History of Suicide Attempt, PTSD, Panic Attacks/Panic Disorder, Alcohol or Substance Dependence, and any combination thereof.

9. A method of determining a subject's predisposition to a mental disorder comprising detecting at least one polymorphism in a gene selected from the group consisting of CACNG2 and Sp4, wherein the polymorphism is selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 and any combination thereof.

10. A method of diagnosing a subject for a mental disorder comprising detecting at least one polymorphism in a gene selected from the group consisting of CACNG2 and Sp4, wherein the polymorphism is selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 and any combination thereof.

11. The method of claim 9 or 10, wherein the polymorphism is detected by a method selected from the group consisting of (a) a primer extension assay; (b) an allele-specific PCR assay; (c) a nucleic acid amplification assay; (d) a hybridization assay; (e) a mismatch-detection assay; (f) an enzymatic nucleic acid cleavage assay; and (g) a sequencing assay.

12. The method of claim 9 or 10, further comprising measuring a clinical symptom of the subject.

13. The method of claim 9 or 10, wherein the polymorphism is in the CACNG2 gene.

14. The method of claim 9 or 10, wherein the polymorphism is in the Sp4 gene.

15. A method of screening for an agent that interacts with an Sp4 or CACNG2 gene product comprising a polymorphism, comprising:
contacting a cell that expresses a polymorphism selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 with a test agent; and
detecting interaction of the agent with the CACNG2 or Sp4 gene product.

16. A method of screening for an agent that inhibits Sp4 or CACNG2 expression or activity comprising:
contacting a cell that expresses a polymorphism selected from the group consisting of rs12673091, rs10245440, rs10261327, rs40245, rs2282888, rs10276352, rs12668354, rs12673091, rs1018954, rs11974306, rs4820239, rs2267341, rs2283981, rs3788521, rs738977, rs738518, rs2009667, rs3484, rs736720, rs2284026 with a test agent; and
detecting a reduction in gene expression or activity.

17. An isolated oligonucleotide comprising a sequence selected from the group consisting of SEQ ID NO:1-37 or 38 and containing nucleotide 11.

18. The isolated oligonucleotide of claim 17, on a solid support.

19. A method of treating a subject with a mental disorder, comprising contacting a subject containing a mutation in a CACNG2 or Sp4 gene with an agent that inhibits production of expression or activity of the mutant gene.

20. A method of treating a subject with a mental disorder, comprising contacting a subject containing a mutation in a CACNG2 or Sp4 gene with a polynucleotide comprising a wild type CACNG2 or Sp4 under conditions wherein the wild type CACNG2 or Sp4 is expressed in vivo.

21. A kit compartmentalized to receive a reagent for measuring mutational burden in the genes of a subject, wherein the reagent comprises an oligonucleotide probe or primer that measures a polymorphism in a gene selected from the group consisting of CACNG2 and/or Sp4.

22. The kit of claim 21, wherein the reagent comprises the oligonucleotide of claim 17 or a solid support of claim 18.
